(19) United States
(12) Patent Application Publication
(10) Pub. No.: US 2025/0000921 A1
(43) Pub. Date: Jan. 2, 2025

(54) Isolated Strain Of Lactic Acid Bacteria For Inhibiting Drug-Resistant Enterobacteriaceae, Lactic Acid Bacterial Composition And Synbiotic Composition Including The Same

(71) Applicant: Jia Jie Biomedical Co. Ltd, Kaohsiung City (TW)

(72) Inventors: Hung-Jen Tang, Tainan City (TW); Chih-Chung Chen, Tainan City (TW); Ying-Chen Lu, Chiayi City (TW); Wen-Fan Hsieh, Kaohsiung City (TW)

(73) Assignee: Jia Jie Biomedical Co., Ltd., Kaohsiung City (TW)

(21) Appl. No.: 18/771,162

(22) Filed: Jul. 12, 2024

Related U.S. Application Data
(63) Continuation of application No. 17/932,427, filed on Sep. 15, 2022.

(30) Foreign Application Priority Data
Jan. 28, 2022 (TW) 111104157
Jan. 28, 2022 (TW) 111104168
Jan. 28, 2022 (TW) 111104175

(51) Int. Cl. A61K 35/747; A61K 31/7016; A61K 31/702; A61P 31/04; C12N 1/20; C12R 1/245; C12R 1/25

(52) U.S. Cl. CPC A61K 35/747; A61K 31/7016; A61K 31/702; A61P 31/04; C12N 1/205; C12R 2001/245; C12R 2001/25

(57) ABSTRACT

The present invention relates to an isolated strain of lactic acid bacteria (LAB) for inhibiting drug-resistant Enterobacteriaceae, in which the isolated strain of the LAB includes Lactobacillus rhamnosus JJ101, Lactobacillus paracasei JJ102 and/or Lactobacillus plantarum JJ103, and the isolated strain of the LAB inhibit growth of the drug-resistant Enterobacteriaceae. After orally administered to a subject, the isolated strain of the LAB can inhibit the growth of the drug-resistant Enterobacteriaceae, and thus can potentially be used to prevent, improve and/or treat the infection of the drug-resistant Enterobacteriaceae.

### CROSS-REFERENCE TO RELATED APPLICATION

This application is a continuation of U.S. application Ser. No. 17/932,427, filed Sep. 15, 2022, which claims priority to Taiwan Application Serial Number 111104175, filed Jan. 28, 2022, Taiwan Application Serial Number 111104168, filed Jan. 28, 2022 and Taiwan Application Serial Number 111104157, filed Jan. 28, 2022, which are herein incorporated by reference in its entirety.

### REFERENCE TO THE SEQUENCE LISTING

A sequence listing is being submitted herein as an xml text file with the name “SP-5661-1-US_SEQ_202409[[LIST]].xml”, created on Aug. 26, 2024, with a file size of 12,288 bytes.

### BACKGROUND

### Field of Invention

The present invention relates to an isolated strain of lactic acid bacteria. More specifically, the present invention relates to an isolated strain of the lactic acid bacteria for inhibiting drug-resistant Enterobacteriaceae, a lactic acid bacterial composition and a synbiotic composition of the same.

### Description of Related Art

Enterobacteriaceae are Gram negative bacteria that belong to the order Enterobacterales of the class Gammaproteobacteria. Enterobacteriaceae are ubiquitous in the environments (e.g., soil and water) as well as in the bodies of organisms (e.g., animal and plant). For example, Enterobacteriaceae are one members of human intestinal tract microbiota. The Enterobacteriaceae include beneficial symbionts as well as opportunistic pathogens. Enterobacteriaceae pathogens can cause diseases such as dysentery, typhoid fever, urinary tract infection, wound infection, liver abscess, septicemia, meningitis and pneumonitis, and Enterobacteriaceae pathogens are members of the most frequent pathogens causing nosocomial and community-onset infections.

Antibiotics are commonly used drugs for treating Enterobacteriaceae infections. Since carbapenem antibiotics process broad spectrum activities and there are few carbapenem-resistant species, the carbapenem antibiotics are the last resorts in treating multidrug-resistant bacteria (MDR) infections at present. However, the Enterobacteriaceae such as Klebsiella pneumoniae has recently evolved ways to reduce the susceptibility to the carbapenem antibiotics. For example, carbapenemase-producing Enterobacteriaceae (CPE) express carbapenemase to degrade the carbapenem antibiotics, thereby increases the morbidity rate and the mortality rate of the infected patients. Thus, the CPE are one of the major global public health threats at present.

In the view of the limitation to control the bacterial infections with the drugs such as the antibiotics, it is necessary to provide a non-drug composition for inhibiting the drug-resistant Enterobacteriaceae to solve the aforementioned problems.

### SUMMARY

Accordingly, one aspect of the present invention is to provide a method of inhibiting drug-resistant Enterobacteriaceae with a synbiotic composition consisting of specific strains of mixed lactic acid bacteria (LABs) and a prebiotic.

According to the aforementioned aspect, the present invention provides a method of inhibiting drug-resistant Enterobacteriaceae including administering a synbiotic composition to a subject in need thereof. The synbiotic composition consists of mixed lactic acid bacteria (LABs) and a prebiotic, the mixed LABs consist of Lactobacillus rhamnosus JJ101, Lactobacillus paracasei JJ102, and Lactobacillus plantarum JJ103. An accession number of the Lactobacillus rhamnosus JJ101 is DSM 34122. An accession number of the Lactobacillus paracasei JJ102 is DSM 34123. An accession number of the Lactobacillus plantarum JJ103 is DSM 34124. The Lactobacillus rhamnosus JJ101, the Lactobacillus paracasei JJ102 and the Lactobacillus plantarum JJ103 are deposited in German Collection of Microorganisms and Cell Cultures (Deutsche Sammlung von Mikroorganismen und Zellkulturen GmbH, DSMZ) on Jan. 12, 2022. The prebiotic consists of isomaltooligosaccharide. The drug-resistant Enterobacteriaceae have KPC-2. Compared to culture solutions obtained by culturing the Lactobacillus rhamnosus JJ101, the Lactobacillus paracasei JJ102, and the Lactobacillus plantarum JJ103 with MRS, respectively, co-culture solutions obtained by subjecting the Lactobacillus rhamnosus JJ101, the Lactobacillus paracasei JJ102, and the Lactobacillus plantarum JJ103 with the MRS having the prebiotic to a co-culture step, respectively, have a lower pH that is less than 5.

In one embodiment of the present invention, a cell ratio of the Lactobacillus rhamnosus JJ101, the Lactobacillus paracasei JJ102 and the Lactobacillus plantarum JJ103 is (1˜5):(1˜5):(1˜10). In one embodiment of the present invention, the synbiotic composition is administered to the subject with an effective dose for 7 days to less than 14 days. In one embodiment of the present invention, the subject is a mouse, and the effective dose is 5.0×10¹⁰ CFU/kg body weight (bw)/day to 1.5×10¹¹ CFU/kg bw/day. In one embodiment of the present invention, the synbiotic composition is an oral composition. In one embodiment of the present invention, an amount of the prebiotic is 1 weight % to 5 weight %.

According to another aspect, the present invention provides a method of inhibiting drug-resistant Enterobacteriaceae comprising administering a synbiotic composition to a subject in need thereof. The synbiotic composition consists of mixed LABs and a prebiotic, in which the mixed LABs consist of Lactobacillus rhamnosus JJ101, Lactobacillus paracasei JJ102 and Lactobacillus plantarum JJ103. An accession number of the Lactobacillus rhamnosus JJ101 is DSM 34122. A n accession number of the Lactobacillus paracasei JJ102 is DSM 34123. A n accession number of the Lactobacillus plantarum JJ103 is DSM 34124. The prebiotic consists of isomaltooligosaccharide and lactulose, and an amount of the prebiotic is 1 weight % to 5 weight %. The drug-resistant Enterobacteriaceae have KPC-2. Compared to culture solutions obtained by culturing the Lactobacillus rhamnosus JJ101, the Lactobacillus paracasei JJ102, and the Lactobacillus plantarum JJ103 with MRS, respectively, co-culture solutions obtained by subjecting the Lactobacillus rhamnosus JJ101, the Lactobacillus paracasei JJ102, and the Lactobacillus plantarum JJ103 with the MRS having the prebiotic to a co-culture step, respectively, have a lower pH that is less than 5.

In one embodiment of the present invention, a cell ratio of the Lactobacillus rhamnosus JJ101, the Lactobacillus paracasei JJ102 and the Lactobacillus plantarum JJ103 can be (1˜5):(1˜5):(1˜10), for example. In one embodiment of the present invention, the synbiotic composition is administered to the subject with an effective dose for 7 days to less than 14 days. In one embodiment of the present invention, the subject is a mouse, and the effective dose can be 5.0×10¹⁰ CFU/kg bw/day to 1.5×10¹¹ CFU/kg bw/day, for example. In one embodiment of the present invention, the synbiotic composition can be an oral composition, for example.

According to the aforementioned aspect, the present invention provides a method of inhibiting drug-resistant Enterobacteriaceae including administering a synbiotic composition to a subject in need thereof. The synbiotic composition consists of mixed LABs, a prebiotic, and additives. The mixed LABs consist of Lactobacillus rhamnosus JJ101, Lactobacillus paracasei JJ102 and Lactobacillus plantarum JJ103. The accession number of the Lactobacillus rhamnosus JJ101 is DSM 34122, an accession number of the Lactobacillus paracasei JJ102 is DSM 34123, an accession number of the Lactobacillus plantarum JJ103 is DSM 34124, the prebiotic consists of isomaltooligosaccharide, the drug-resistant Enterobacteriaceae have KPC-2, and compared to culture solutions obtained by culturing the Lactobacillus rhamnosus JJ101, the Lactobacillus paracasei JJ102, and the Lactobacillus plantarum JJ103 with MRS, respectively, co-culture solutions obtained by subjecting the Lactobacillus rhamnosus JJ101, the Lactobacillus paracasei JJ102, and the Lactobacillus plantarum JJ103 to a co-culture step with the MRS having the prebiotic, respectively, have a lower pH that is less than 5.

In one embodiment of the present invention, a cell ratio of the Lactobacillus rhamnosus JJ101, the Lactobacillus paracasei JJ102 and the Lactobacillus plantarum JJ103 is (1˜5):(1˜5):(1˜10). In one embodiment of the present invention, the synbiotic composition is administered to the subject with an effective dose for 7 days to less than 14 days. In one embodiment of the present invention, the subject is a mouse, and the effective dose is 5.0×10¹⁰ CFU/kg body weight (bw)/day to 1.5×10¹¹ CFU/kg bw/day. In one embodiment of the present invention, the synbiotic composition is an oral composition. In one embodiment of the present invention, the additives is selected from the group consisting of solvents, emulsifiers, suspending agents, disintegrating agents, binders, stabilizers, chelating agents, diluents, gelling agents, preservatives, lubricants, absorption delaying agents, and any combination thereof. In one embodiment of the present invention, an amount of the prebiotic is 1 weight % to 5 weight %. In one embodiment of the present invention, a dosage form of the synbiotic composition is an aqueous solution, a suspension, a dispersion, a emulsion, a hydrogel, a gel, a solid lipid nanoparticle, a lozenge, a granule, a powder or a capsule.

By applying the synbiotic composition, growth of the drug-resistant Enterobacteriaceae having KPC-2 can be inhibited in vitro and/or in vivo, indicating that the synbiotic composition of the present invention have potentials to prevent, improve and/or treat drug-resistant Enterobacteriaceae infections.

### BRIEF DESCRIPTION OF THE DRAWINGS

The invention can be more fully understood by reading the followed detailed description of the embodiment, with reference made to the accompanying drawings as follows:

FIG. 1 was a line graph illustrating the amount of the Lactobacillus rhamnosus in mouse feces after the oral administration of the Lactobacillus rhamnosus to mice was conducted for 3 consecutive days and stopped in accordance with one embodiment of the present invention.

FIG. 2 was a line graph illustrating the amount of the Lactobacillus paracasei in mouse feces after the oral administration of the Lactobacillus paracasei to mice was conducted for 3 consecutive days and stopped in accordance with one embodiment of the present invention.

FIG. 3 was a line graph illustrating the amount of the Lactobacillus plantarum in mouse feces after the oral administration of the Lactobacillus plantarum to mice was conducted for 3 consecutive days and stopped in accordance with one embodiment of the present invention.

FIG. 4 was a line graph illustrating the CPE amount in the feces of the infected mice in accordance with one embodiment of the present invention.

FIG. 5 was a line graph illustrating the CPE amount in the feces of the infected mice of different groups in accordance with one embodiment of the present invention.

FIGS. 6A and 6B were line graphs of the CPE amount and the pH values of the co-culture solutions containing different prebiotics after the strain JJ101 and the CPE were co-cultured in according to one embodiment of the present invention.

FIGS. 7A and 7B were line graphs of the CPE amount and the pH values of the co-culture solutions containing different prebiotics after the strain JJ102 and the CPE were co-cultured in according to one embodiment of the present invention.

FIGS. 8A and 8B were line graphs of the CPE amounts and the pH values of the co-culture solutions containing different prebiotics after the strain JJ103 and the CPE were co-cultured in according to one embodiment of the present invention.

FIG. 9 was a line graph of CPE amounts of the feces of the infected mice of different groups according to one embodiment of the present invention.

### DETAILED DESCRIPTION

As mentioned above, the present invention provides an isolated strain of lactic acid bacteria (LAB) for inhibiting drug-resistant Enterobacteriaceae, a lactic acid bacterial composition and a synbiotic composition of the same, in which the isolated strain of the LAB includes Lactobacillus rhamnosus JJ101. As in vitro co-cutured experiments and animal experiments have proven, the isolated strain of the LAB is able to inhibit the growth of the drug-resistant Enterobacteriaceae.

The term “lactic acid bacteria (LAB)” mentioned herein refers the bacteria that can produce lactic acid and/or acetic acid by degrading sugars (e.g., lactose, glucose, sucrose and/or fructose), such as Lactobacillus, Pediococcus, Bacillus and Bifidobacterium. It is noted that some LAB strains may interfere with other LAB strains and affect their functions, but combinations of specific strains may function synergistically, thereby improving the retention ability and/or the functions of the strains in animal bodies (i.e., in intestinal tract). Thus, to select a single LAB strain or multiple LAB strains (called mixed LABs) should depend on the strain, the subject and/or the functions when applying the LAB.

It should be supplemented that, a better retention ability of the LAB in animal bodies (i.e., intestinal tract) refers to a longer retention time and/or more viable cells of the LAB inhabiting in animal bodies (i.e., intestinal tract), in which the viable cells of the LAB can be evaluated by calculating the viable cells per unit weight of the feces, for example. In one embodiment, since processing acid tolerance and salinity tolerance, LAB has better retention abilities in the intestinal tract. Note that Lactobacillus rhamnosus, Lactobacillus paracasei, Lactobacillus plantarum are also called Lacticaseibacillus rhamnosus, Lacticaseibacillus paracasei, Lactiplantibacillus plantarum, respectively.

In one embodiment, the isolated strain of the LAB can be selected from a group consisting of Lactobacillus rhamnosus, Lactobacillus paracasei, Lactobacillus plantarum and any combination thereof. In one embodiment, the Lactobacillus rhamnosus can be the Lactobacillus rhamnosus JJ101 with the accesstion number of DSM 34122 (also referred to as strain JJ101), the Lactobacillus paracasei can be the Lactobacillus paracasei JJ102 with the accesstion number of DSM 34123 (also referred to as strain JJ102), and the Lactobacillus plantarum can be the Lactobacillus plantarum JJ103 with the accesstion number of DSM 34124 (also referred to as strain JJ103). It should be supplemented that, the Lactobacillus rhamnosus JJ101, the Lactobacillus paracasei JJ102 and the Lactobacillus plantarum JJ103 are depostied in Leibniz Institute DSMZ-German Collection of Microorganisms and Cell Cultures GmbH (address: Inhoffernstraβe 7B, 38124 Braunschweig, Germany) on Jan. 12, 2022 and have been tested viability on Jan. 18, 2022.

In one embodiment, a cell ratio of the Lactobacillus rhamnosus JJ101, the Lactobacillus paracasei JJ102 and the Lactobacillus plantarum JJ103 can be 1˜5:1˜5:1˜10, for example, so that the Lactobacillus rhamnosus JJ101, the Lactobacillus paracasei JJ102 and the Lactobacillus plantarum JJ103 can function synergistically in animal bodies, thereby inhibiting the growth of the drug-resistant Enterobacteriaceae more efficiently. In one specific embodiment, the cell ratio of the Lactobacillus rhamnosus JJ101, the Lactobacillus paracasei JJ102 and the Lactobacillus plantarum JJ103 can be 1:1:1, for example.

As proved by animal experiments, compared to other strains of the same species, after orally administered to animals for 3 consecutive days, any one of the Lactobacillus rhamnosus JJ101, the Lactobacillus paracasei JJ102 and the Lactobacillus plantarum JJ103 have more viable cells retained in the intestinal tract, indicating that the Lactobacillus rhamnosus JJ101, the Lactobacillus paracasei JJ102 and the Lactobacillus plantarum JJ103 have better retention abilities in the intesinal tract.

The term “drug-resistant Enterobacteriaceae” mentioned herein refers to the strains of the Enterobacteriaceae having resistance to antibiotics. The term “LAB inhibits drug-resistant Enterobacteriaceae” refers that after the drug-resistant Enterobacteriaceae have been co-cultured with the LAB in vitro, the growth of the drug-resistant Enterobacteriaceae can be inhibited effectively (e.g., a decrease of at least two orders of magnitude in cell numbers, i.e., 99% inhibition), or after the LAB are orally administered to an animal, the amount of the drug-resistant Enterobacteriaceae in the animal body decreases (e.g., after the administration of LAB for at least 7 consecutive days, the drug-resistant Enterobacteriaceae in the feces of the infected animal has a decrease of at least five orders of magnitude in cell numbers, i.e., 99.999% inhibition). It should be supplemented that, the inhibition can be the percentage of the difference of the initial bacterial numbers and the treated bacterial numbers over the initial bacterial numbers, in which the initial bacterial numbers are the numbers of the viable cells of the drug-resistant Enterobacteriaceae before being co-cultured with the LAB, and the treated bacterial numbers are the numbers of the viable cells of the drug-resistant Enterobacteriaceae after being co-cultured with the LAB. In another embodiment, the initial bacterial numbers is the amount of the drug-resistant Enterobacteriaceae in the feces of the infected animal before being oral administered with LAB, and the treated bacterial number is the amount of the drug-resistant Enterobacteriaceae in the feces of the infected animal after being orally administered with the LAB.

In one embodiment, the aforementioned antibiotics can be β-lactam antibiotics, for example. The β-lactam antibiotics can inhibit the growth of the bacteria by interfering cell wall synthesis. The β-lactam antibiotics can include but not limited to penicillin, cephalosporin, carbapenem and monobactam. In one embodiment, the drug-resistant Enterobacteriaceae can be β-lactam-resistant Enterobacteriaceae, for example. In one embodiment, the drug-resistant Enterobacteriaceae can be carbapenem-resistant Enterobacteriaceae (CRE), for example. In some specific embodiments, the drug-resistant Enterobacteriaceae can be carbapenemase-producing Enterobacteriaceae (CPE), for example.

In addition, the carbapenemase is a kind of β-lactamases that have the ability to hydrolyze the β-lactam antibiotics (e.g., carbapenemase), thereby decreasing the susceptibility of CPE to the β-lactam antibiotics. Klebsiella pneumoniae carbapenemase (KPC), one of the carbapenemases, was named due to its first identification in Klebsiella pneumoniae in 1996. The genes of KPCs are located at plasmids, so KPCs can transform from species to species. Up to now, strains of other Enterobacteriaceae (e.g., Citrobacter freundii, Escherichia coli, Enterobacter gergoviae, Enterobacter aerogenes, Enterobacter cloacae, Klebsiella oxytoca, Proteus mirabilis, Salmonella enterica and Serratia marcescens) and other Gram-negative bacteria that are not Enterobacteriaceae (e.g., Pseudomonas aeruginosa, Pseudomonas putida, Acinetobacter sp.) have been found to produce KPCs. KPCs can be classified into KPC-1, KPC-2, KPC-3, etc., according to their gene sequences. Clinically, the drug-resistant Enterobacteriaceae containing KPC-2 [such as the Klebsiella pneumoniae with sequence type (ST) 11] are more common than those containing other KPCs.

The animal experiments have proven that after the oral administration of any one of the Lactobacillus rhamnosus JJ101, the Lactobacillus paracasei JJ102 or the Lactobacillus plantarum JJ103 to the CPE-infected animals for at least 4 days, the amount of the drug-resistant Enterobacteriaceae in the feces of the infected animal can be effectively decreased, and after the administration of the aforementioned strains for at least 7 days, the amount of the drug-resistant Enterobacteriaceae decreases at least two orders of magnitude, i.e., 99% inhibition, indicating that all of the Lactobacillus rhamnosus JJ101, the Lactobacillus paracasei JJ102 and the Lactobacillus plantarum JJ103 have the effects to inhibit the growth of the drug-resistant Enterobacteriaceae. Besides, after the oral administration of any one of the Lactobacillus rhamnosus JJ101, the Lactobacillus paracasei JJ102 or the Lactobacillus plantarum JJ103 at once for at least 7 days, the amount of the drug-resistant Enterobacteriaceae decreases at least three orders of magnitude, i.e., 99.9% inhibition, indicating that when using the 3 strains together, the effect to inhibit the growth of the drug-resistant Enterobacteriaceae is better.

In one embodiment, the isolated strain of the LAB can be cultured with prebiotics, thereby forming synbiotics. The term “prebiotics” mentioned herein refers to the material that cannot be digested by the host, but can improve the host's health by benefiting the growth and/or the metabolic activities of specific strains in the host's digestive tract.

Common prebiotics include disaccharide, oligosaccharide carbohydrates (OSCs), resistant starch and other materials that are not sugar. The specific examples of prebiotics can be fructo-oligosaccharide, galacto-oligosaccharide, polydextrose, xylo-oligosaccharide, fructooligosaccharide, isomalto-oligosaccharides, lactulose and inulin, etc. In one embodiment, the prebiotics can include but not limited to lactulose and/or isomalto-oligosaccharides. In one embodiment, lactulose and/or isomalto-oligosaccharides can promote the LAB to produce acidic materials (e.g., organic acid), thereby promoting the ability of the LAB to inhibit the growth of the drug-resistant Enterobacteriaceae.

As proven by in vitro co-culture experiments, lactulose and/or isomalto-oligosaccharides can promote any one of the Lactobacillus rhamnosus JJ101, the Lactobacillus paracasei JJ102 and the Lactobacillus plantarum JJ103to produce acidic materials, so as to obtain a co-culture solution with pH less than 5, thereby inhibiting the growth of the drug-resistant Enterobacteriaceae. In addition, as proved by animal experiments, the amount of the drug-resistant Enterobacteriaceae in the digestive tract of the infected animals administered with the synbiotics decreases faster than that of the infected animals administered with mixed LABs (without prebiotics), in which the synbiotics contain the mixed LABs and the prebiotic. For example, after both the mixed LABs and the prebiotics are administered for 7 days, the amount of the drug-resistant Enterobacteriaceae in the digestive track of the infected animal decreases at least five orders of magnitude, i.e., 99.999% inhibition.

In one embodiment, there is no limitation on the amount of the prebiotics not exceeding the safe dosage. The daily safe dosage of the prebiotics for an adult can be lower than 10 g, for example, to avoid unpleasant symptoms such as bloating and diarrhea. In one embodiment, the amount of the prebiotics can be 1 weight % to 5 weight %, 1.5 weight % to 2.5 weight % or 2 weight %, for example, so that the growth and/or the metabalic activities of the LAB can be fully stimulated, but the amount of the prebiotics will not exceed the aforementioned daily safe dosage.

When applying the aforementioned LAB, there is no specific limitation on the route of administration, and can be adjusted depending on actual needs. The amount and the frequency of administering the aforementioned LAB can be adjusted flexibly depending on needs. In one embodiment, the in vitro effective dose of the Lactobacillus rhamnosus JJ101, the Lactobacillus paracasei JJ102and the Lactobacillus plantarum JJ103 in culture solutions in vitro is 10⁵ CFU/mL to 10⁷ CFU/mL. In one embodiment, when the subject is a mouse, the effective dose of the LAB can be 5.0×10¹⁰ CFU/kg body weight/day to 1.5×10¹¹ CFU/kg body weight/day. For example, in the aforementioned animal experiment, the effective dose of the LAB to the mouse is 1.0×10¹¹ CFU/kg bw/day, which is equivalent to 2.0×10⁹ CFU/mouse (20 g bw)/day.

It should be supplemented that, in animal experiments, since the drug-resistant Enterobacteriaceae are orally administered to a mouse, the amount of the drug-resistant Enterobacteriaceae in the digestive tract of the mouse is much higher than that in the digestive tract of a clinical patient. Moreover, the mouse has a feces-eating habit, so that the mouse will ingest the drug-resistant Enterobacteriaceae in the feces repeatedly. Thus, the mouse requires the oral administration with a higher dosage of the isolated strain of the LAB to decrease the amount of the drug-resistant Enterobacteriaceae effectively. In other words, for clinical application, an effective dose of the LAB for an adult to effectively inhibit the amount of the drug-resistant Enterobacteriaceae is lower than the effective amount of the LAB for the mouse in the animal experiment. In a specific embodiment, the effective amount of the isolated strain of the LAB to an adult can be 1.0×10⁸ CFU/60 kg bw/day to 1.0×10¹⁰ CFU/60 kg bw/day. In one embodiment, the isolated strain of the LAB is administered to a subject for several consecutive days with the aforementioned effective dose. In one embodiment, the isolated strain of the LAB is administered to the subject for at least 7 consecutive days, e.g., 7 days to 1 year, or 14 days to 6 months.

The aforementioned isolated strain of the LAB can inhibit the growth of the drug-resistant Enterobacteriaceae, so that the isolated strain of the LAB can be an active ingredient of a lactic acid bacterial composition and/or a synbiotic composition. In one embodiment, the lactic acid bacterial composition and/or the synbiotic composition can be an oral composition, for example. In one embodiment, the lactic acid bacterial composition and/or the synbiotic composition can be a food composition or a medical composition. In one embodiment, the lactic acid bacterial composition and/or the synbiotic composition can selectively include food-or drug-acceptable carriers, excipients, diluents, adjuvants and/or additives, such as solvents, emulsifiers, suspending agents, disintegrating agents, binders, stabilizers, chelating agents, diluents, gelling agents, preservatives, lubricants and/or absorption delaying agents. In one embodiment, there is no special limit on the dosage form of the lactic acid bacterial composition and/or the synbiotic composition, and can be a aqueous solution, a suspension, a dispersion, a emulsion (single-phase or multi-phase dispersion systems, single-or multi-chamber liposomes), a hydrogel, a gel, a solid lipid nanoparticle, a lozenge, a granule, a powder or a capsule, etc.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the followed claims.

### EXAMPLE 1

### Isolation, Culture and Microorganism Traits of Isolated Strains of LAB

The following 11 isolated strains of the LAB, LYC1504, JJ101, LYC1119, JJ102, LYC1129, LYC1031, LYC1112, LYC1117, LYC1146, LYC1159 and JJ103, were isolated from fruit ferments. The 11 isolated strains of the LAB were inoculated on de Man, Rogosa and Sharpe (MRS) agar media by quadrant streak technique, followed by incubation at 37° C. for 16 hours to 18 hours respectively, so as to obtain single colonies. Then, the single colonies were inoculated in MRS broth, followed by incubation at 37° C. for 16 hours to 24 hours, so as to obtain LAB culture solutions. The LAB culture solutions were centrifuged to obtain pellets.

The pellets were subjected to RNA purification, reverse transcription and polymerase chain reaction (PCR) with an upstream primer and a downstream primer to obtain a nucleic acid fragment of 16S rDNA, followed by nucleic acid sequencing to obtain the nucleic acid sequence of the 16S rDNA. It is noted that the nucleic acid sequence of the upstream primer was shown as SEQ ID NO: 1, and the nucleic acid sequence of the downstream primer was shown as SEQ ID NO: 2. Among the 11 isolated strains of LAB, 2 of them were identified as Lactobacillus rhamnosus (the strains LYC1504 and JJ101), 3 of them were identified as Lactobacillus paracasei (the strains LYC1119, JJ102 and LYC1129), and 6 of them were identified as Lactobacillus plantarum (the strains LYC1031,LYC1112, LYC1117, LYC1146, LYC1159 and JJ103) by aligning with Basic Local Alignment Search Tool (BLAST).

The nucleic acid sequence of the 16S rDNA of the aforementioned strain JJ101 was shown as SEQ ID NO:3. The nucleic acid sequence of the 16S rDNA of the aforementioned strain JJ102 was shown as SEQ ID NO:4. The nucleic acid sequence of the 16S rDNA of the aforementioned strain JJ103 was shown as SEQ ID NO:5. The strains JJ101, JJ102 and JJ103 were deposited in DSMZ on Jan. 12, 2022 and tested viability on Jan. 18, 2022, in which the accession number of the strain JJ101 was DSM 34122, the accession number of the strain JJ102 was DSM 34123, and the accession number of the strain JJ103 was DSM 34124. The strains JJ101, JJ102 and JJ103 were also deposited at BCRC on Dec. 22, 2021, in which the accession number of the strain JJ101 was BCRC 911088, the accession number of the strain JJ102 was BCRC 911089, and the accession number of the strain JJ103 was BCRC 911090.

It is noted that the strain JJ101 (Lactobacillus rhamnosus) had milky white, opaque, sphericla, entire colonies with smooth protruded surfaces, in which the cells had a short rod-like shape with rounded ends and appeared in forms of single, pairs, a short-chain or a chain. The cells of the strain JJ101 did not form flagellum nor spore and was immobile. The cells of the strain JJ101 were tested positive by Gram's stain. The strain JJ102 (Lactobacillus paracasei) had milky white, opaque, near-spherical or spherical, entire colonies with smooth protruded surfaces, in which the cells had a short rod-like shape with rounded ends and appeared in forms of single, pairs or a short-chain. The cells of the strain JJ102 did not form flagellum nor spore and was immobile. The cells of the strain JJ102 were tested positive by Gram's stain. The strain JJ103 (Lactobacillus plantarum) had milky white, opaque, spherical to a bit irregular, entire colonies with smooth protruded surfaces, in which the cells were straight and in a rod shape with rounded ends and appeared in forms of single, pairs or a short-chain. The cells of the strain JJ103 did not form flagellum nor spore but was mobile. The cells of the strain JJ103 were tested positive by Gram's stain.

### EXAMPLE 2

### Evaluation of Retention Abilities of LAB and Drug-Resistant Enterobacteriaceae in Animal Body

### 1. The Retention Abilities of the LAB in an Animal Body

The BALB/c mice (abbreviate as mice below) were used as experimental animals. Female mice at 5 weeks of age were kept in independent aerated cages in an animal room, so that the mice could adapt to the environment. During the period for adaptation, the mice could consume standard chow and sterilized distilled water ad libitum. In the animal room, the temperature was 23+3° C., the relative humidity was 60+10%, and there was a 12 hours light period and a 12 hour dark period every day. The mice were subjected to the following evaluation at 6 weeks of age.

First, the mice were administered with antibiotics, and the amounts of the bacteria in the mouse feces were detected, so as to confirm whether the feces were germ-free. The method to detect the amount of the bacteria was described as the following. The fresh mouse feces were weighted, followed by grinding with addition of 1 mL normal saline (NS) to form test solutions. Then, the test solutions were spread on Enterobacter medium, Mueller Hinton broth (MHB) agar medium and LAB medium, followed by incubation at 37° C. for 24 hours for colony count. The aforementioned Enterobacter medium used for detecting Enterobacter was an eosin methylene blue agar containing 16 μg/ml vancomycin, 64 μg/mL ampicillin and 16 μg/mL cefotaxime. The LAB medium used for detecting LAB was a MRS agar containing 32 μg/mL vancomycin, and the pH value of the LAB medium was 5.0.

After the mouse feces were detected and confirmed to be germ-less, the mice were gavaged with different LAB solutions, in which the LAB solutions were obtained by re-dissolving the LAB pellets of the aforementioned 11 strains of the LAB in phosphate buffered saline (PBS) respectively. The amounts of the LAB in the LAB solutions were adjusted to orally administer 2.0×10⁹ CFU/day LAB to the mice for consecutive 3 days. Then, the gavage was stopped, and the aforementioned LAB medium was used to detect LAB amounts in the mouse feces 1, 3 and 7 days after the gavage was stopped. The LAB amounts (unit: CFU/g) were the ratio value of the numbers of the viable LAB cells to the weight of the mouse feces.

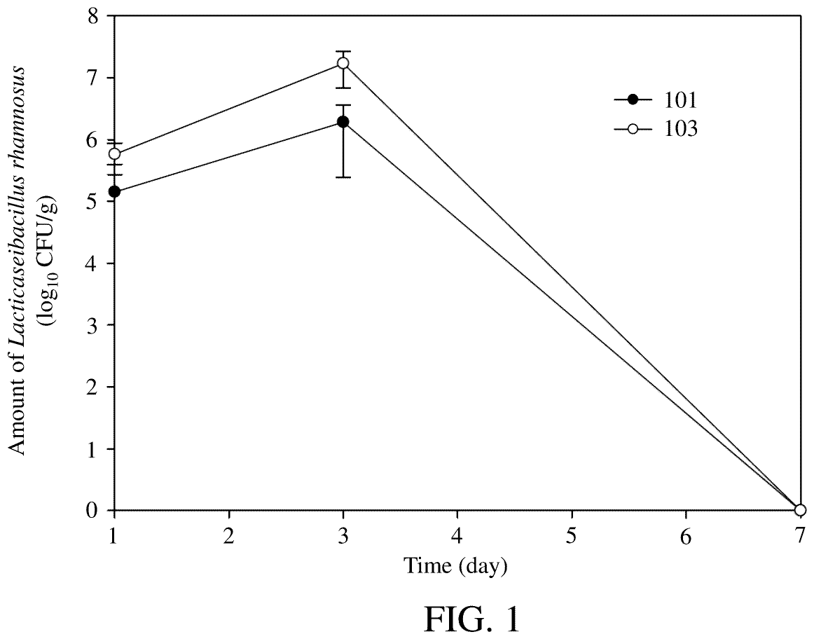

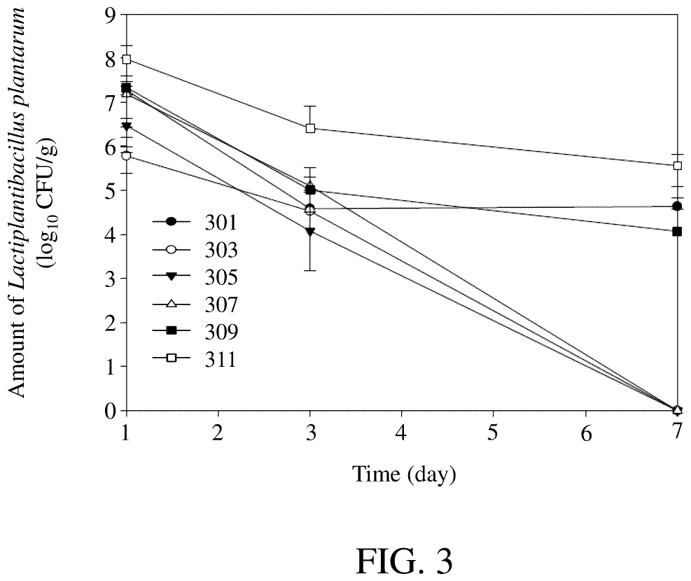

Referring to FIG. 1, in which FIG. 1 was a line graph illustrating the amount of the Lactobacillus rhamnosus in mouse feces after the oral administration of the Lactobacillus rhamnosus to mice was conducted for 3 consecutive days and stopped in accordance with one embodiment of the present invention. The x-axis represented time (unit: day), the y-axis represented the amount of the Lactobacillus rhamnosus (unit: CFU/g), and lines 101 and 103 represented the strains LYC1504 and JJ101, respectively. as showed in FIGS. 1, 1 and 3 days after the gavage was stopped, the amount of the strain JJ101 (line 103) in the mouse feces was higher than the amount of the strain LYC1504 (line 101), in which the amount of the strain JJ101 was higher than 10⁷ CFU/g 3 days after the gavage was stopped, indicating that the strain JJ101 had a better retention ability.

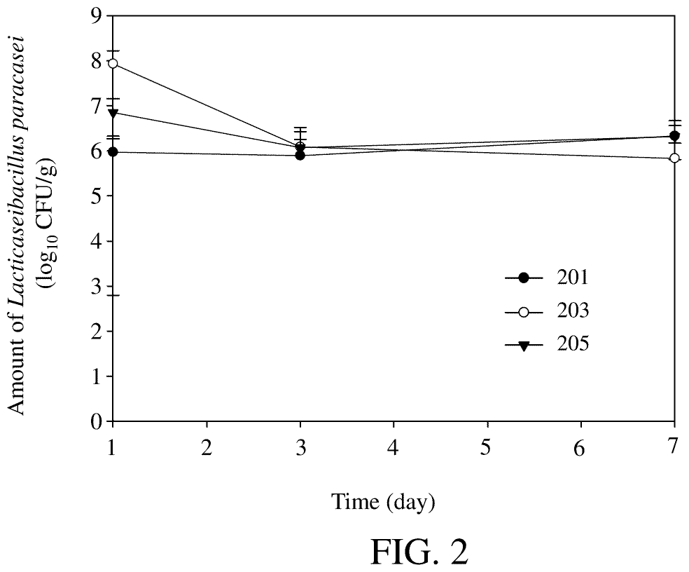

Referring to FIG. 2, in which FIG. 2 was a line graph illustrating the amount of the Lactobacillus paracasei in mouse feces after the oral administration of the Lactobacillus paracasei to mice was conducted for 3 consecutive days and stopped in accordance with one embodiment of the present invention. The x-axis represented time (unit: day), the y-axis represented the amount of the Lactobacillus paracasei (unit: CFU/g), and lines 201, 203 and 205 represented the strains LYC1119, JJ102 and LYC1229, respectively. as showed in FIG. 2, 1 day after the gavage was stopped, the amount of the strain JJ102 (line 203) in the mouse feces was higher than that of the strains 1119 and LYC1229 (lines 201, 205), in which the amount of the strain JJ102 was higher than 10⁷ CFU/g, indicating that the strain JJ102 had a better retention ability.

Referring to FIG. 3, in which FIG. 3 was a line graph illustrating the amount of the Lactobacillus plantarum in mouse feces after the oral administration of the Lactobacillus plantarum to mice was conducted for 3 consecutive days and stopped in accordance with one embodiment of the present invention. The x-axis represented time (unit: day), the y-axis represented the amount of the Lactobacillus plantarum (unit: CFU/g), and lines 301, 303, 305, 307, 309 and 311 represented the strains LYC1031, LYC1112, LYC1117, LYC1146, LYC1159 and JJ103, respectively.

As showed in FIGS. 3, 1, 3 and 7 days after the gavage was stopped, the amount of the strain JJ103 (line 311) in the mouse feces was higher than that of other strains (lines 301 to 309), in which 3 days after the gavage, the amount of the strain JJ103 was 10⁶ CFU/g to 10⁷ CFU/g, and 7 days after the gavage, the amount of the strain JJ103 was still higher than 10⁵ CFU/g, proving that the strain JJ103 had a better retention ability compared to other Lactobacillus plantarum.

### 2. The Retention Abilities of the Drug-Resistant Enterobacteriaceae in an Animal Body

Strains KPC001, KPC011, KPC021 and KPC035 were KPC-2 expressing drug-resistant Enterobacteriaceae (abbreviated as CPE in the following) isolated from Medical Research Department, Clinical Trial Center, Chi Mei Medical Center. The CPE were inoculated on the Enterobacter medium by the quadrant streak technique and were incubated at 37° C. for 16 hours to 18 hours, so as to obtain single colonies. Then, the single colonies were inoculated in the MHB and incubated 37° C. for 16 hours to 24 hours, thereby obtaining CPE culture solutions. The CPE culture solutions were centrifuged to obtain CPE pellets.

The antibiotics were administered to the mice everyday till the mouse feces were germ-less. Then, the mice were gavaged with CPE solutions, in which the CPE solutions were obtained by re-dissolving the CPE pellets in a water solution containing 20 weight % skim milk powders. The amounts of the CPE in the CPE solutions were adjusted to orally administer 3.0×10⁸ CFU/day of the CPE to the mice for consecutive 3 days, thereby obtaining infected mice. After that, the gavage was stopped, and the mouse feces were collected once again on 1, 2, 7, 10, 14, 17, 21, 24, 28, 31 and 35 days after the gavage was stopped. The CPE amount (unit: CFU/g) in the feces were detected by using the MHB agar, in which the CPE amount was the ratio value of the number of the viable CPE cells to the feces weight.

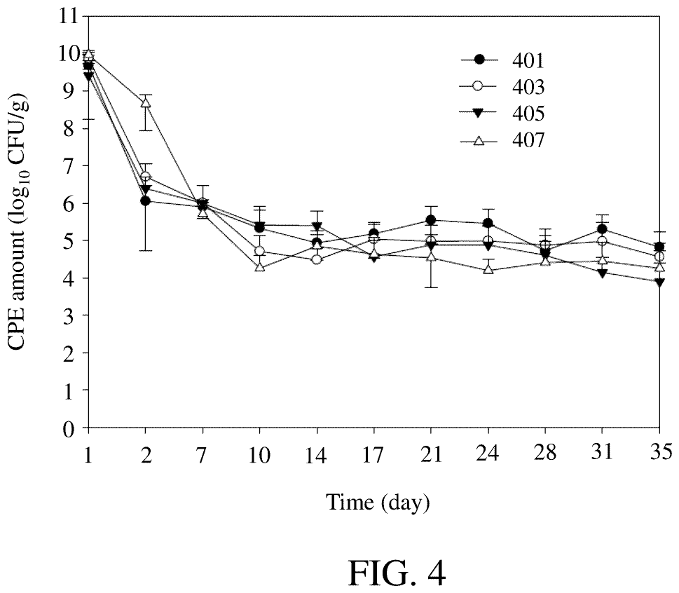

Referring to FIG. 4, in which FIG. 4 was a line graph illustrating the CPE amount in the feces of the infected mice in accordance with one embodiment of the present invention. The x-axis represented time (unit: day), and the y-axis represented the CPE amount (unit: CFU/g), line 401, line 403, line 405 and line 407 represented the strains KPC001, KPC011, KPC021 and KPC035, respectively. As shown in FIG. 4, 1 day after the gavage was stopped, the amount of different strains of the CPE in the mouse feces were all about 10¹⁰ CFU/g, and 4 to 35 days after the gavage was stopped, the amount of different CPE strains in the mouse feces were still kept at 10⁴ to 10⁶ CFU/g. The aforementioned result showed that there were no differences among the retention abilities of different strains of CPE. The following evaluation was performed with the strain KPC001.

### EXAMPLE 3

### Evaluation of Ability to Inhibit Drug-Resistant Enterobacteriaceae of LAB

The antibiotics were administered to the mice everyday till the mouse feces were germ-less. Then, 3.0×10⁸ CFU/day of the CPE were orally administered to the mice to obtain infected mice. After that, the CPE amount in the feces of the infected mouse was detected to obtain the CPE amount of the infected mice not orally administered with the LAB. Next, the infected mice were divided into 4 groups (blank group, experimental groups 1, 2, 3 and 4). The infected mice of the blank group were orally administered with PBS for 21consecutive days, the infected mice of the experimental group 1 were orally administered with 2.0×10⁹ CFU/day of the strain JJ101 for 21 consecutive days, the infected mice of the experimental group 2 were orally administered with 2.0×10⁹ CFU/day of the strain JJ102 for 21 consecutive days, the infected mice of the experimental group 3 were orally administered with 2.0×10⁹ CFU/day of the strain JJ103 for 21 consecutive days, and the infected mice of the experimental group 4 were orally administered with 2.0×10⁹ CFU/day of mixed LABs for 21 consecutive days, in which the mixed LABs were consisted of the strains JJ101, JJ102 and JJ103 with a cell ratio of 1:1:1. The CPE amount in the feces of the infected mouse was detected on 4, 7, 11, 14, 18 and 21 days after the mice were orally administered with the LAB.

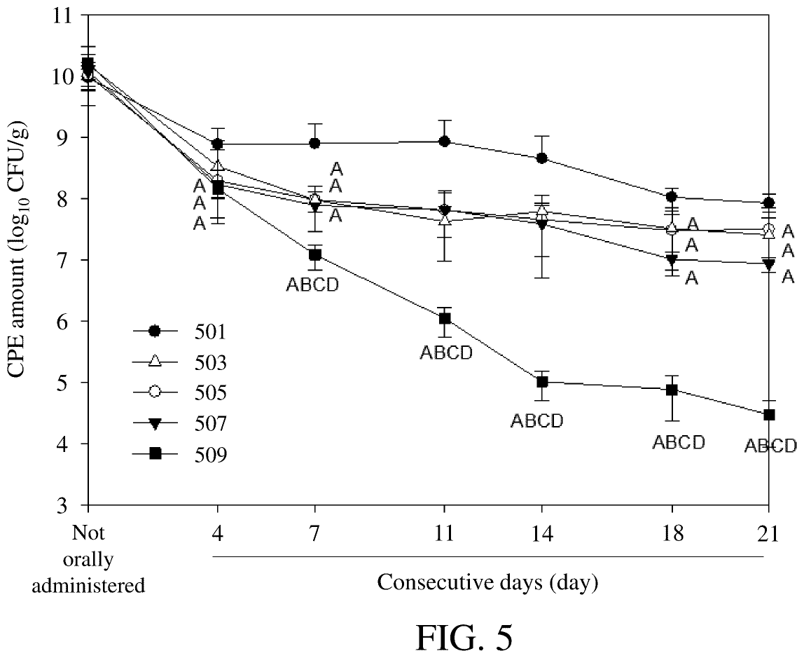

Referring to FIG. 5, in which FIG. 5 was a line graph illustrating the CPE amount in the feces of the infected mice of the different groups in accordance with one embodiment of the present invention. The x-axis represented the consecutive days (unit: day) of the oral administration of the LAB to the infected mice, the y-axis represented the CPE amount (unit: CFU/g) in the feces of the infected mice, lines 501, 503, 505, 507 and 509 represented the blank group, the experimental groups 1, 2, 3 and 4, and different letters A, B, C and D represented there were statistically significant differences (p<0.05).

As shown in FIG. 5, the CPE amount in the feces of the infected mice of the experimental groups 1 to 4 (lines 503 to 509) is lower than that of the blank group (line 501), indicating that the individual or the combination of the strains JJ101, JJ102 and JJ103 could inhibit the growth of the CPE. Moreover, compared to the CPE amount of the infected mice not orally administered with the LAB, the CPE amounts in the feces of the infected mice of the experimental group 4 deceased at least five orders of magnitude (i.e., 99.999% inhibition) after 21 consecutive days of oral administration of the mixed LABs to the infected mice. However, the CPE amounts in the feces of the infected mice of the experimental groups 1 to 3 deceased two to three orders of magnitude merely (i.e., 99% to 99.9% inhibition) after 21 consecutive days of oral administration of the mixed LABs to the infected mice, indicating that the mixed LABs had a better ability to inhibit the growth of the CPE when compared to that of the single strains of the LAB.

### EXAMPLE 4

### Evaluation of Ability to Inhibit Drug-Resistant Enterobacteriaceae of Synbiotics

### 1. Abilities of the Prebiotics to Promote the LAB to Produce Acidic Materials

LAB could produce acidic materials (e.g., lactic acid and/or acetic acid) by degrading sugar, so as to decline the pH values of the environment (e.g., intestinal tract), thereby inhibiting CPE. Therefore, if the LAB could use prebiotics more efficiently, the ability to inhibit the growth of the CPE would be better with the combination of the prebiotics and LAB.

The aforementioned 11 strains of the LAB were inoculated in the MRS broth without glucose made by different recipes, followed by incubation at 37° C. for 24 hours to obtain cultures. Then, the pH values of the cultures were measured, and the results (average +standard deviation of 3 repeats) were recorded in Table 1, in which the group NON represents the MRS broth without sugar, the group SUC represents the MRS broth containing 2 weight % of sucrose, the group FOS represents the MRS broth containing 2 weight % of fructo-oligosaccharide, the group IN represents the MRS broth containing 2 weight % of inulin, the group IMO represents the MRS broth containing 2 weight % of isomalto-oligosaccharides, the group LU represents the MRS broth containing 2 weight % of lactulose, and the group XOS represents the MRS broth containing 2 weight % of xylo-oligosaccharide.

TABLE 1

| | pH value | | | | |
|---|---|---|---|---|---|
| | Lacticaseibacillus rhamnosus | | Lacticaseibacillus paracasei | | |
| Group | JJ101 | LYC1504 | JJ102 | LYC1119 | LYC1229 |
| NON | 6.08 ± 0.02 | 6.13 ± 0.02 | 6.12 ± 0.01 | 6.20 ± 0.00 | 6.14 ± 0.00 |
| SUC | 5.31 ± 0.01 | 5.29 ± 0.01 | 3.82 ± 0.03 | 5.47 ± 0.02 | 4.34 ± 0.02 |
| FOS | 5.89 ± 0.07 | 5.85 ± 0.03 | 3.68 ± 0.04 | 4.10 ± 0.03 | 4.07 ± 0.02 |
| IN | 5.40 ± 0.01 | 5.41 ± 0.03 | 3.70 ± 0.05 | 4.09 ± 0.02 | 4.04 ± 0.04 |
| XOS | 5.21 ± 0.01 | 5.18 ± 0.02 | 5.02 ± 0.03 | 5.56 ± 0.02 | 5.34 ± 0.00 |
| LU | 3.78 ± 0.02 | 3.83 ± 0.01 | 3.94 ± 0.04 | 4.36 ± 0.05 | 4.37 ± 0.03 |
| IMO | 4.29 ± 0.01 | 4.28 ± 0.01 | 4.60 ± 0.02 | 5.10 ± 0.02 | 4.61 ± 0.02 |

| | pH value | | | | | |
|---|---|---|---|---|---|---|
| | Lactiplantibacillus plantarum | | | | | |
| Group | JJ103 | LYC1031 | LYC1112 | LYC1117 | LYC1146 | LYC1159 |
| NON | 6.21 ± 0.00 | 6.19 ± 0.00 | 6.06 ± 0.01 | 6.15 ± 0.00 | 6.13 ± 0.01 | 6.19 ± 0.01 |
| SUC | 3.82 ± 0.05 | 3.83 ± 0.05 | 3.95 ± 0.04 | 4.01 ± 0.04 | 3.99 ± 0.04 | 3.98 ± 0.05 |
| FOS | 5.94 ± 0.01 | 5.94 ± 0.01 | 5.81 ± 0.01 | 3.78 ± 0.06 | 3.69 ± 0.06 | 3.70 ± 0.06 |
| IN | 5.03 ± 0.01 | 4.97 ± 0.01 | 4.88 ± 0.03 | 3.77 ± 0.04 | 3.80 ± 0.05 | 3.72 ± 0.04 |
| XOS | 5.17 ± 0.01 | 5.15 ± 0.01 | 5.09 ± 0.02 | 5.09 ± 0.02 | 5.19 ± 0.02 | 5.18 ± 0.02 |
| LU | 3.78 ± 0.04 | 3.74 ± 0.04 | 3.79 ± 0.06 | 3.82 ± 0.03 | 3.72 ± 0.04 | 3.76 ± 0.04 |
| IMO | 4.57 ± 0.03 | 4.48 ± 0.02 | 4.37 ± 0.03 | 3.91 ± 0.05 | 4.58 ± 0.03 | 4.45 ± 0.03 |

As shown in FIG. 1, the pH values of the cultures in the groups SUC, FOS, IN, XOS, IMO and LU were lower than that of the group NON, indicating that sugar could promote the LAB to produce acidic materials. Further, the pH values of the strain JJ101 cultures were lower than 5.0 in the groups LU and IMO, indicating that lactulose and isomalto-oligosaccharides had the abilities to promote the strain JJ101 to product acidic materials. The pH values of the strain JJ102 cultures were higher than 5.0 only in the group XOS, indicating that among the sugars, only xylo-oligosaccharide could not promote the strain JJ102 to produce acidic materials. The pH values of the strain JJ103 cultures was lower than 5.0 in the groups SUC, LU and IMO, indicating that sucrose, lactulose and isomalto-oligosaccharides had the abilities to promote the strain JJ103 to product acidic materials. It was noted that sucrose could not be used as prebiotics since sucrose could be digested by animals. Thus, when the mixed LABs were consisting of the strain JJ101, JJ102 and JJ103, lactulose and/or isomalto-oligosaccharides should be selected as prebiotics.

### 2. The pH Values and the Ability to Inhibit the Growth of the CPE of the Synbiotics Formed by the Strain JJ101 and Different Prebiotics

Among each aforementioned species of the LAB (Lactobacillus rhamnosus, Lactobacillus paracasei and Lactobacillus plantarum), the single strains of the LAB with better retention abilities (i.e., the strains JJ101, JJ102 and JJ103) were added to pH 6.5 co-cultured solutions with the CPE (the strain KPC001), so as to perform a co-culture trail, in which the initial LAB amounts were 10⁷ CFU/mL, and the initial CPE amounts were 10⁶ CFU/mL in the co-cultured solutions. Next, the co-cultured solutions were subjected to LAB amount detection, CPE amount detection and pH value detection, so as to obtain initial LAB amounts, initial CPE amounts and pH values (i.e., incubated for 0 hour). The LAB amount detection was conducted by spreading the co-cultured solutions on MRS agar media with pH 5.5, followed by incubation at 37° C. to obtain single colonies of the LAB. The LAB amounts (unit: CFU/mL) of the co-cultured solutions could be calculated with the numbers of the single colonies of the LAB. The CPE amount detection was conducted by spreading the co-cultured solutions on EMB agar media with 16 μg/mL ampicillin, followed by incubation at 37° C. to obtain single colonies of the CPE. The CPE amounts (unit: CFU/mL) of the co-cultured solutions could be calculated with the numbers of the single colonies of the CPE.

The co-cultured solutions were prepared by the MRS broth without glucose and MHB with a volume ratio of 1:1. Sugars were added to the co-cultured solutions or not according to the corresponding groups, in which the co-cultured solutions in the group NON contained no sugar, the co-cultured solutions in the group SUC contained 2 weight % sucrose, the co-cultured solutions in the group FOS contained 2 weight % fructo-oligosaccharide, the co-cultured solutions in the group IN contained 2 weight % inulin, the co-cultured solutions in the group XOS contained 2 weight % xylo-oligosaccharide, the co-cultured solutions in the group LU contained 2 weight % lactulose, and the co-cultured solutions in the group IMO contained 2 weight % isomalto-oligosaccharides.

The co-cultured solutions were incubated at 37° C., and the LAB amount detection, the CPE amount detection and the pH value detection were performed at the 3, 6, 24, and 48 hours of the incubation.

In the co-cultured experiment that the aforementioned strain JJ101 and CPE were co-cultured in different co-cultured solutions, the results of the LAB amount detection showed that after 48 hours of incubation, the amounts of the strain JJ101 in the co-cultured solutions in the groups SUC, FOS, IN, XOS, LU and IMO were higher than 1.0×10⁸ CFU/mL and lower than 1.0×10⁹ CFU/mL, which were higher than that of the group NON (not shown in the figures), indicating that prebiotics benefited growth of the strain JJ101 in vitro.

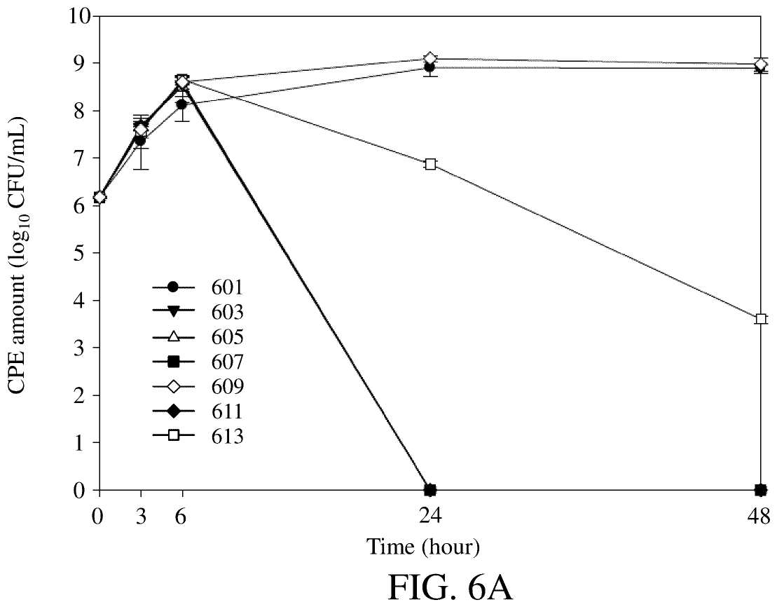

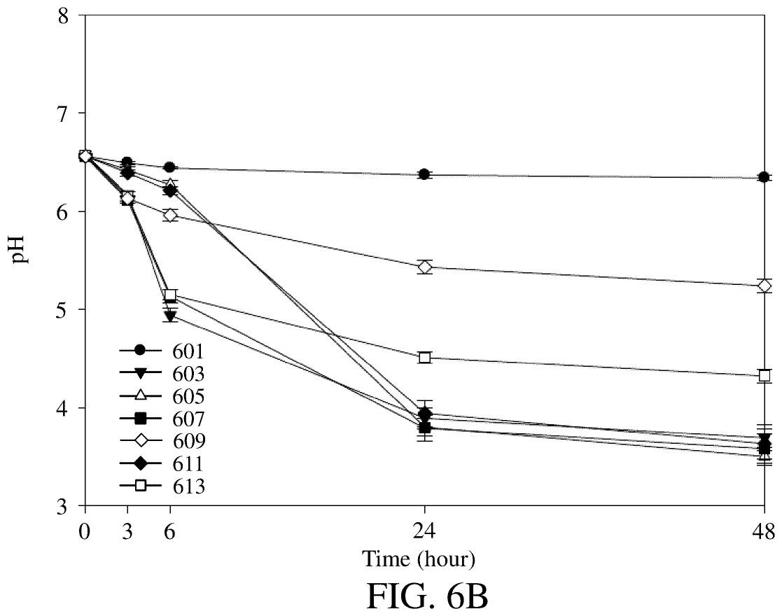

Referring to FIGS. 6A and 6B, in which FIGS. 6A and 6B were line graphs of the CPE amount (FIG. 6A) and the pH values (FIG. 6B) of the co-culture solutions containing different prebiotics after the strain JJ101 and the CPE were co-cultured in according to one embodiment of the present invention. In FIG. 6A, the x-axis represented time (unit: hour), and the y-axis represented CPE amount (unit: CFU/mL). In FIG. 6B, the x-axis represented time (unit: hour), and the y-axis represented pH value. In FIGS. 6A and 6B, lines 601, 603, 605, 607, 609, 611 and 613 represented the groups NON, SUC, FOS, IN, XOS, LU and IMO, respectively.

As shown in FIG. 6A, after incubating for 24 hours, the CPE amounts in the co-culture solutions of the groups SUC, FOS, IN and LU (lines 603 to 607 and 611) were lower than the detection limit. After incubating for 48 hours, the CPE amount in the co-culture solution of the group IMO (line 613) was two orders of magnitude lower than the initial CPE amount (0 hour), i.e., 99% inhibition. However, after incubating for 48 hours, the CPE amounts in the co-culture solutions of the groups NON and XOS (lines 601 and 609) were higher than the initial CPE amount (0 hour). as shown in FIG. 6B, after incubating for 24 to 48 hours, the pH values of the co-cultured solutions were lower than 5 in the groups SUC, FOS, IN, LU and IMO (lines 603, 605, 607, 611 and 613), but the pH values of the co-cultured solutions were higher than 5 in the groups XOS and NON (lines 609 and 601). The aforementioned results proved that sucrose, fructo-oligosaccharide, inulin, lactulose and isomalto-oligosaccharides could promote the strain JJ101 to produce acidic materials, so that the pH values of the co-cultured solutions were lower than 5, thereby inhibiting growth of the CPE.

### 3. The pH Values and the Ability to Inhibit the Growth of the CPE of the Synbiotics Formed by the Strain JJ102 and Different Prebiotics

In the co-cultured experiment which the aforementioned strain JJ102 and the CPE were co-cultured in different co-cultured solutions, the results of the LAB amount detection showed that after 48 hours of incubation, the amounts of the strain JJ102 in the co-culture solutions in the in the groups SUC to XOS and LU to IMO were higher than 1.0×10⁸ CFU/mL and lower than 1.0×10⁹ CFU/mL (not shown in the figures), but was higher than the group NON, indicating that the prebiotics benefited growth of the strain JJ102 in vitro.

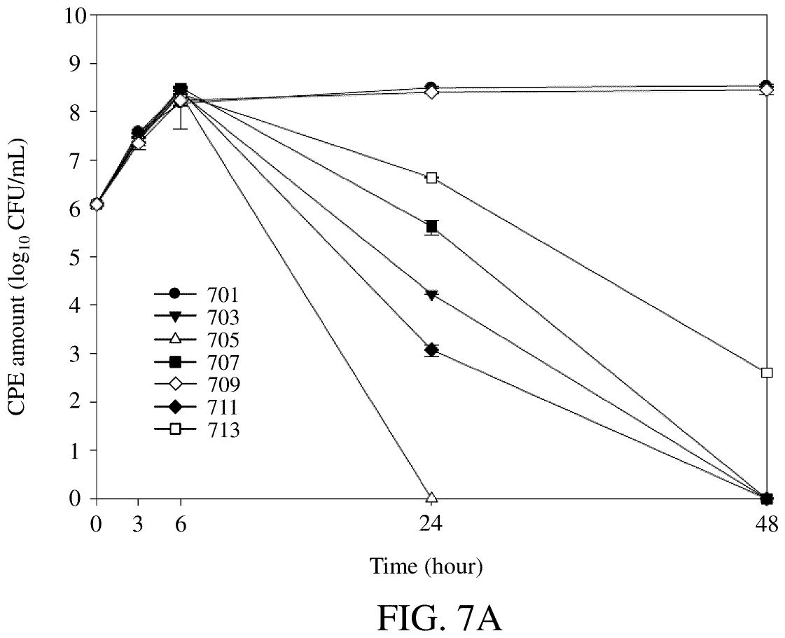

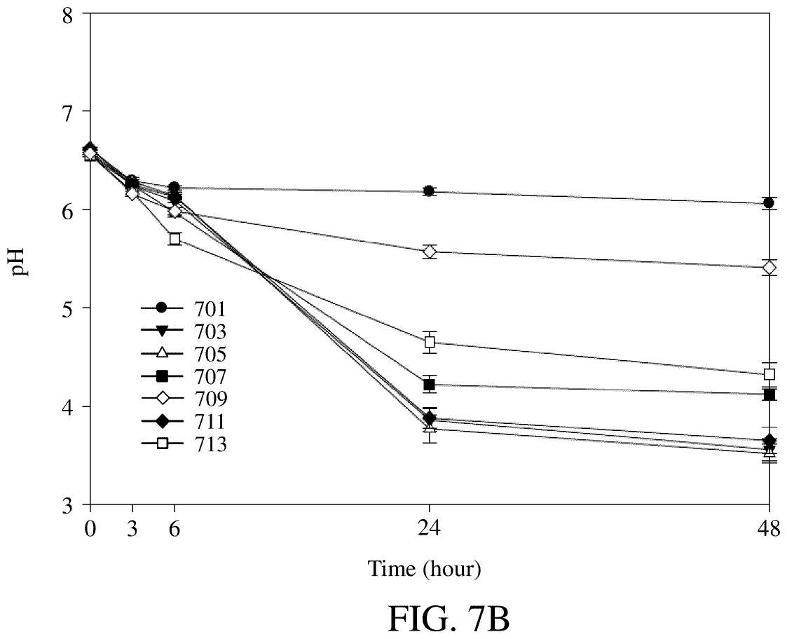

Referring to FIGS. 7A and 7B, in which FIGS. 7A and 7B were line graphs of the CPE amount (FIG. 7A) and the pH values (FIG. 7B) of the co-culture solutions containing different prebiotics after the strain JJ102 and the CPE were co-cultured in according to one embodiment of the present invention. In FIG. 7A, the x-axis represented time (unit: hour), and the y-axis represented CPE amount (unit: CFU/mL). In FIG. 7B, the x-axis represented time (unit: hour), and the y-axis represented pH value. In FIGS. 7A and 7B, lines 701, 703, 705, 707, 709, 711 and 713 represented the groups NON, SUC, FOS, IN, XOS, LU and IMO, respectively.

As shown in FIG. 7A, after incubating for 24 hours, the CPE amount in the co-culture solution in the group FOS (line 705) was lower than the detection limit. After incubating for 48 hours, the CPE amounts in the co-culture solutions in the groups SUC, IN and LU (line 703, 707 and 711) were lower than the detection limit. The CPE amount in the co-culture solution in the group IMO (line 713) was three orders of magnitude lower than the initial CPE amount (0 hour), i.e., 99.9% inhibition. After incubating for 48 hours, the CPE amounts in the co-culture solutions in the groups NON and XOS (lines 701 and 709) were higher than the initial CPE amount (0 hour). In FIG. 7B, after incubating for 48 hours, the pH values of the co-cultured solutions were higher than 5 in the groups XOS and NON (line 709 and 701), but the pH values of the co-cultured solutions were lower than 5 in the groups SUC, FOS, IN, LU and IMO. The aforementioned results proved that sucrose, fructo-oligosaccharide, inulin, lactulose and isomalto-oligosaccharides could promote the strain JJ102 to produce acidic materials, so that the pH values of the co-culture solutions were lower than 5, thereby inhibiting growth of the CPE.

### 4. The pH Values and the Ability to Inhibit the Growth of the CPE of the Synbiotics Formed by the Strain JJ103 and Different Prebiotics

The results of the LAB amount detection showed that after incubating for 48 hours, the amount of the strain JJ103 (9.0×10⁸ CFU/mL) in the co-cultured solution of the group NON was the least, and the amounts of the strain JJ103 in the co-cultured solution of the groups SUC to XOS and LU to IMO were higher than 9.0×10⁸ CFU/mL (not shown in the figures), proving that prebiotics benefited growth of the strain JJ103 in vitro.

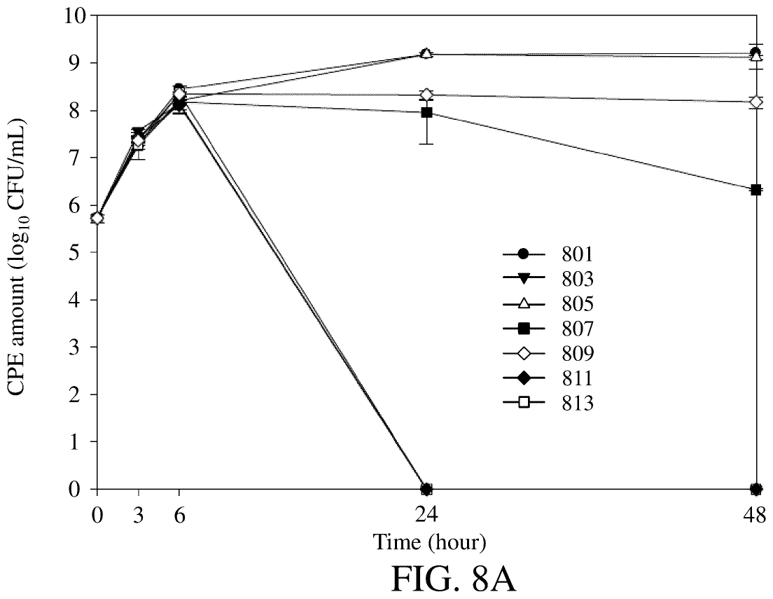

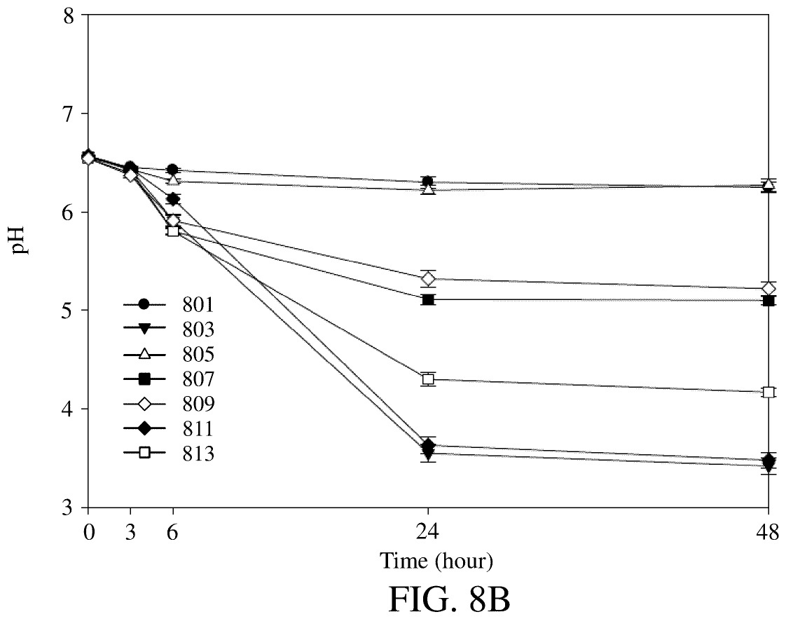

Referring to FIGS. 8A and 8B, in which FIGS. 8A and 8B were line graphs of the CPE amount (FIG. 8A) and the pH values (FIG. 8B) of the co-culture solutions containing different prebiotics after the strain JJ103 and CPE were co-cultured in according to one embodiment of the present invention. In FIG. 8A, the x-axis represented time (unit: hour), and the y-axis represented CPE amount (unit: CFU/mL). In FIG. 8B, the x-axis represented time (unit: hour), and the y-axis represented pH value. In FIGS. 8A and 8B, lines 801, 803, 805, 807, 809, 811 and 813 represented the groups NON, SUC, FOS, IN, XOS, LU and IMO, respectively.

As shown in FIG. 8A, after incubating for 24 hours, the CPE amounts in the co-culture solutions of the groups SUC, LU and IMO (line 803, 811 and 813) were lower than the detection limit, but the CPE amounts in the co-culture solutions of the groups NON, FOS, IN and XOS were higher than the initial CPE amount. As shown in FIG. 8B, the pH values of the co-cultured solutions were smaller than 5 in the groups SUC, LU and IMO (lines 803, 811 and 813), but the pH values of the co-cultured solutions were higher than 5 in the groups NON, FOS, IN and XOS. Moreover, sucrose, lactulose and isomalto-oligosaccharides were prove to be able to promote the strain JJ103 to produce acidic materials, so that the pH values of the co-cultured solutions was lower than 5, thereby inhibiting growth of the CPE.

As shown in FIGS. 6A, 6B, 7A, 7B, 8A and 8B, sucrose, fructo-oligosaccharide, inulin, lactulose and isomalto-oligosaccharides were able to effectively promote the strain JJ101 and the strain JJ102 to produce acidic materials, but among them, only sucrose, lactulose and isomalto-oligosaccharides were able to effectively promote the strain JJ103 to produce acidic materials, in which sucrose could be digestible for animals, so that lactulose and isomalto-oligosaccharides was used in the subsequent experiment as prebiotics.

### 5. The Growth of the CPE of the Synbiotics Formed by Mixed LABs and Different Prebiotics

The pellets of the strains JJ101, JJ102 and JJ103 were re-dissolved with PBS to obtain mixed LABs solutions, in which the cell ratio of the strains JJ101, JJ102 and JJ103 was 1:1:1. Then, lactulose synbiotics were obtained by adjusted 2 weight % lactulose in mixed LABs solutions, and the isomalto-oligosaccharides synbiotics were obtained by adjusted 2 weight % isomalto-oligosaccharides in mixed LABs solutions.

The mice were divided into 4 groups (blank group, control group, experimental groups 1 and 2) and were orally administered with antibiotics every day till the mouse feces were germ-less. Next, the infected mice were obtained by orally administered 3.0×10⁸ CFU/day of the CPE for 3 consecutive days. Then, the CPE amounts of the infected mice not orally administered with the LAB were obtained by detecting the CPE amounts in the feces of the infected mice. After that, the infected mice in the blank group were orally administered with PBS for 21 consecutive days, the infected mice in the control group were orally administered with the mixed LABs solutions for 21 consecutive days, the infected mice of the experimental group 1 were orally administered with the lactulose synbiotics for 21 consecutive days, and the infected mice of the experimental group 2 were orally administered with the isomalto-oligosaccharides synbiotics for 21 consecutive days. At the time when the mice were orally administered with the LAB for 4, 7, 11, 14, 18 consecutive days, the CPE amount in the mouse feces were detected. In addition, the cells of the mixed LABs were 2.0×10⁹

CFU/day for oral administration to the infected mouse of the control group, the experimental groups 1 and 2.

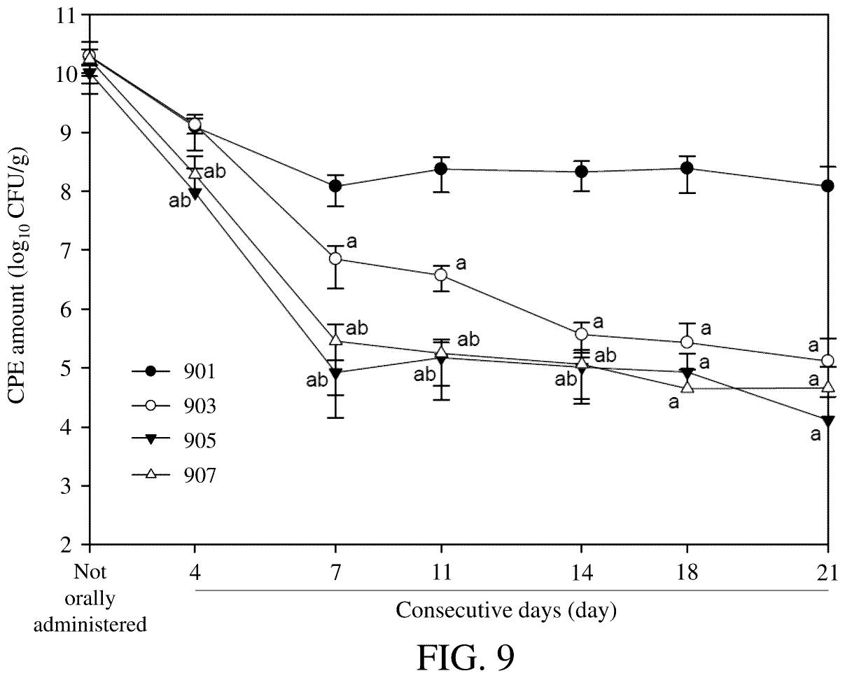

Referring to FIG. 9, in which was a line graph of the CPE amounts of the feces of the infected mice of the different groups according to one embodiment of the present invention. The x-axis represented the consecutive days (unit: day) that the mice were orally administrated with the LAB, the y-axis represented the CPE amount (unit: CFU/g) in the feces of the infected mice, and lines 901, 903, 905 and 907 represented the blank group, the control group, the experimental groups 1 and 2, respectively, and different letters a and b represented there were statistically significant differences in different groups (p<0.05).

As shown in FIG. 9, after the mice were orally administered with the PBS, the mixed LABs or the synbiotics for 21 consecutive days, the CPE amount in the feces of the infected mouse of the control group, the experimental groups 1 and 2 were significantly lower than that of the blank group, proving that the mixed LABs, the lactulose synbiotic and the isomalto-oligosaccharides synbiotic could inhibit the growth activity of the CPE. However, after the mice were orally administered with the PBS, the mixed LABs or the synbiotics for 7 consecutive days, the CPE amount in the feces of the infected mouse in the experimental groups 1 and 2 were significantly lower than that of the control group, proving that the lactulose synbiotic and/or the isomalto-oligosaccharides synbiotic were able to decrease the CPE amount faster than the mixed LABs (not containing prebiotics).

In sum, the isolated strain of the LAB such as Lactobacillus rhamnosus JJ101, Lactobacillus paracasei JJ102 and Lactobacillus plantarum JJ103 can inhibit the growth activity of the drug-resistant Enterobacteriaceae, indicating that these isolated strains of LAB have the potential to prevent, improve and/or treat the drug-resistant Enterobacteriaceae infection.

Noting that although the specific isolated strains of the LAB, specific methods, specific effective dose, specific effective method of the administration, specific models and specific evaluation method are shown in the present invention as examples to explain the LAB, the lactic acid bacterial composition and the synbiotic composition including the same of the present invention, it will be apparent to those skilled in the art that the present invention is not limited to what have mentioned. Without departing from the scope or spirit of the invention, it is intended that other strains of the LAB, other methods, other effective dose, other effective method of administration, other models and other evaluation method can also explain the present invention.

Although the present invention has been described in considerable detail with reference to certain embodiments thereof, other embodiments are possible. Therefore, the spirit and scope of the appended claims should not be limited to the description of the embodiments contained herein.

What is claimed is:

1. A method of inhibiting drug-resistant Enterobacteriaceae comprising administering a synbiotic composition to a subject in need thereof, wherein the synbiotic composition consists of mixed lactic acid bacteria (LABs) and a prebiotic, the mixed LABs consist of Lactobacillus rhamnosus JJ101, Lactobacillus paracasei JJ102, and Lactobacillus plantarum JJ103, an accession number of the Lactobacillus rhamnosus JJ101 is DSM 34122, an accession number of the Lactobacillus paracasei JJ102 is DSM 34123, an accession number of the Lactobacillus plantarum JJ103 is DSM 34124, the Lactobacillus rhamnosus JJ101, the Lactobacillus paracasei JJ102 and the Lactobacillus plantarum JJ103 are deposited in German Collection of Microorganisms and Cell Cultures (Deutsche Sammlung von Mikroorganismen und Zellkulturen GmbH, DSMZ) on Jan. 12, 2022, the prebiotic consists of isomaltooligosaccharide, the drug-resistant Enterobacteriaceae have KPC-2, and compared to culture solutions obtained by culturing the Lactobacillus rhamnosus JJ101, the Lactobacillus paracasei JJ102, and the Lactobacillus plantarum JJ103 with MRS, respectively, co-culture solutions obtained by subjecting the Lactobacillus rhamnosus JJ101, the Lactobacillus paracasei JJ102, and the Lactobacillus plantarum JJ103 to a co-culture step with the MRS having the prebiotic, respectively, have a lower pH that is less than 5.

2. The method according to claim 1, wherein a cell ratio of the Lactobacillus rhamnosus JJ101, the Lactobacillus paracasei JJ102 and the Lactobacillus plantarum JJ103 is (1˜5):(1˜5):(1˜10).

3. The method according to claim 1, wherein the synbiotic composition is administered to the subject with an effective dose for 7 days to less than 14 days.

4. The method according to claim 3, wherein the subject is a mouse, and the effective dose is 5.0×10¹⁰ CFU/kg body weight (bw)/day to 1.5×10¹¹ CFU/kg bw/day.

5. The method according to claim 1, wherein the synbiotic composition is an oral composition.

6. The method according to claim 1, wherein an amount of the prebiotic is 1 weight % to 5 weight %.

7. A method of inhibiting drug-resistant Enterobacteriaceae comprising administering a synbiotic composition to a subject in need thereof, wherein the synbiotic composition consists of mixed LABs and a prebiotic, the mixed LABs consist of Lactobacillus rhamnosus JJ101, Lactobacillus paracasei JJ102 and Lactobacillus plantarum JJ103, an accession number of the Lactobacillus rhamnosus JJ101 is DSM 34122, an accession number of the Lactobacillus paracasei JJ102 is DSM 34123, an accession number of the Lactobacillus plantarum JJ103 is DSM 34124, the prebiotic consists of isomaltooligosaccharide and lactulose, an amount of the prebiotic is 1 weight % to 5 weight %, the drug-resistant Enterobacteriaceae have KPC-2, and compared to culture solutions obtained by culturing the Lactobacillus rhamnosus JJ101, the Lactobacillus paracasei JJ102, and the Lactobacillus plantarum JJ103 with MRS, respectively, co-culture solutions obtained by subjecting the Lactobacillus rhamnosus JJ101, the Lactobacillus paracasei JJ102, and the Lactobacillus plantarum JJ103 to a co-culture step with the MRS having the prebiotic, respectively, have a lower pH that is less than 5.

8. The method according to claim 7, wherein a cell ratio of the Lactobacillus rhamnosus JJ101, the Lactobacillus paracasei JJ102 and the Lactobacillus plantarum JJ103 is (1˜5):(1˜5):(1˜10).

9. The method according to claim 7, wherein the synbiotic composition is administered to the subject with an effective dose for 7 days to less than 14 days.

10. The method according to claim 9, wherein the subject is a mouse, and the effective dose is 5.0×10¹⁰ CFU/kg body weight (bw)/day to 1.5×10¹¹ CFU/kg bw/day.

11. The method according to claim 7, wherein the synbiotic composition is an oral composition.

12. A method of inhibiting drug-resistant Enterobacteriaceae comprising administering a synbiotic composition to a subject in need thereof, wherein the synbiotic composition consists of mixed LABs, a prebiotic, and additives, the mixed LABs consist of Lactobacillus rhamnosus JJ101, Lactobacillus paracasei JJ102 and Lactobacillus plantarum JJ103, an accession number of the Lactobacillus rhamnosus JJ101 is DSM 34122, an accession number of the Lactobacillus paracasei JJ102 is DSM 34123, an accession number of the Lactobacillus plantarum JJ103 is DSM 34124, the prebiotic consists of isomaltooligosaccharide, the drug-resistant Enterobacteriaceae have KPC-2, and compared to culture solutions obtained by culturing the Lactobacillus rhamnosus JJ101, the Lactobacillus paracasei JJ102, and the Lactobacillus plantarum JJ103 with MRS, respectively, co-culture solutions obtained by subjecting the Lactobacillus rhamnosus JJ101, the Lactobacillus paracasei JJ102, and the Lactobacillus plantarum JJ103 to a co-culture step with the MRS having the prebiotic, respectively, have a lower pH that is less than 5.

13. The method according to claim 12, wherein a cell ratio of the Lactobacillus rhamnosus JJ101, the Lactobacillus paracasei JJ102 and the Lactobacillus plantarum JJ103 is (1˜5):(1˜5):(1˜10).

14. The method according to claim 12, wherein the synbiotic composition is administered to the subject with an effective dose for 7 days to less than 14 days.

15. The method according to claim 14, wherein the subject is a mouse, and the effective dose is 5.0×10¹⁰ CFU/kg body weight (bw)/day to 1.5×10¹¹ CFU/kg bw/day.

16. The method according to claim 12, wherein the synbiotic composition is an oral composition.

17. The method according to claim 12, wherein the additives is selected from the group consisting of solvents, emulsifiers, suspending agents, disintegrating agents, binders, stabilizers, chelating agents, diluents, gelling agents, preservatives, lubricants, absorption delaying agents, and any combination thereof.

18. The method according to claim 12, wherein an amount of the prebiotic is 1 weight % to 5 weight %.

19. The method according to claim 12, wherein a dosage form of the synbiotic composition is an aqueous solution, a suspension, a dispersion, a emulsion, a hydrogel, a gel, a solid lipid nanoparticle, a lozenge, a granule, a powder or a capsule.


SEQUENCE LISTING (5 sequences)

SEQ ID NO: 1 - DNA, 20 bp, synthetic construct
  source 1..20: mol_type = other DNA; note = Up stream primer
  misc_difference 12: note = m is a or c
agagtttgatcmtggctcag

SEQ ID NO: 2 - DNA, 22 bp, synthetic construct
  source 1..22: mol_type = other DNA; note = Down stream primer
  misc_difference 6: note = y is t or c
tacggytaccttgttacgactt

SEQ ID NO: 3 - DNA, 1423 bp, Lactobacillus rhamnosus
  source 1..1423: mol_type = genomic DNA; note = 16s rRNA of Lactobacillus rhamnosus JJ101
agttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagcc

SEQ ID NO: 4 - DNA, 1434 bp, Lactobacillus paracasei
  source 1..1434: mol_type = genomic DNA; note = 16s rRNA of Lactobacillus paracasei JJ102
agtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgt

SEQ ID NO: 5 - DNA, 1432 bp, Lactobacillus plantarum
  source 1..1432: mol_type = genomic DNA; note = 16s rRNA of Lactobacillus plantarum JJ103
agtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcc